(19) United States
(12) Patent Application Publication
(10) Pub. No.: US 2021/0220373 A1
(43) Pub. Date: Jul. 22, 2021

(54) ORALLY DISINTEGRATING PHARMACUTICAL COMPOSITION COMPRISING NEFOPAM AND PROCESS FOR PREPARING THE SAME

(71) Applicant: ATHENA PHARMACEUTIQUES SAS, Louveciennes (FR)

(72) Inventor: Mahendra B. Chaudhari, Maharashtra (IN)

(21) Appl. No.: 17/149,127

(22) Filed: Jan. 14, 2021

(30) Foreign Application Priority Data
Jan. 21, 2020 (IN) 202011002685

(51) Int. Cl. A61K 31/395; A61K 9/16; A61K 9/00; A61K 9/20

(52) U.S. Cl. CPC A61K 31/5545; A61K 9/1635; A61K 9/0056; A61K 9/2095; A61K 9/2018; A61K 9/2059; A61K 9/2027; A61K 9/2054

(57) ABSTRACT

The present invention relates to an orally disintegrating pharmaceutical composition comprising Nefopam and pharmaceutically acceptable inert excipients and a process or preparing the same. The composition comprises 5 to 10% w/w of Nefopam with respect to weight of the composition

### CROSS REFERENCE TO RELATED APPLICATION

This application claims priority to and the benefit of Indian Application No. 202011002685, filed Jan. 21, 2020. The related application is incorporated herein in its entirety by reference.

### FIELD OF THE INVENTION

The present invention relates to an orally disintegrating pharmaceutical composition comprising Nefopam and pharmaceutically acceptable inert excipients and a process or preparing the same.

### BACKGROUND OF THE INVENTION

Nefopam is a centrally acting non-narcotic analgesic primarily used to treat moderate to severe, acute or chronic pain. Chemically it is 5-methyl-1-phenyl-3, 4, 5, 6-tetrahydro-1H-2, 5-benzoxazocine hydrochloride with following chemical structure.

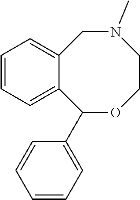 [SMILES: CN1CCOC(c2ccccc2)c2ccccc2C1]

It is available commercially in UK as a film coated tablet available in 30 mg strength. It is approved for the relief of acute and chronic pain, including post-operative pain, dental pain, musculo-skeletal pain, acute traumatic pain and cancer pain. The commercially available product tablet core constituents are: Calcium hydrogen phosphate dehydrate, Microcrystalline cellulose, Starch, pregelatinised, Magnesium stearate, Hydrogenated vegetable oil, Silica, colloidal anhydrous. The Film-coat constituents are Hypromellose (E464), Titanium dioxide (E171), Lactose monohydrate, Macrogol 3000, Triacetin.

US application US20110275626 discloses a formulation for oral transmucosal administration of at least one active principle for treating a spastic crisis, including: at least one active principle present in base form and/or in salt form, chosen from peripheral action antispasmodics and nefopam; an aqueous alcohol solution titrating at least 35° alcohol; and optionally, another active principle present in base form and/or in salt form, chosen from central action analgesics; the active principle(s) being present in a state of stable and complete dissolution in the aqueous alcohol solution.

Orally disintegrating tablets (ODTs) differ from conventional tablets in that they are designed to be dissolved/disintegrated in an oral cavity rather than swallowed whole. Accordingly, ODTs have been developed such that the same medicine can be orally administered in a simple and effective manner to children, adults and people with impaired swallowing function and similar conditions. Even a patient who is not suffering from any swallowing limitation will find it much easier to take an ODT as compared to a conventional tablet. An additional reason to use an orally disintegrating tablet is the convenience of a tablet that can be taken without water. In view of ease of administration, the ODTs have a rapid disintegration rate as compared to conventional tablets.

H Y Guan; Preparation of Nefopam Hydrochloride Orally Disintegrating Tablet, a Chinese thesis discloses preparation of the tablets formulated according to the orthogonal design by direct compression. β-cyclodextrin inclusion complex techniques was evaluated to mask the bitter taste of nefopam hydrochloride. The stability was investigated through accelerated test. The in vivo behavior of the tablets was evaluated in beagle dogs after administration of orally disintegrating tablets and ordinary tablets in the market by a randomized, crossover and self-control trial. Pharmacokinetic parameters of the formulations were compared, and the relative bioavailability and bioequivalence were calculated.

There is a need of orally disintegrating tablet composition comprising Nefopam hydrochloride to provide quick relief of the acute pain. Accordingly, the inventors of the present invention have developed orally disintegrating pharmaceutical composition comprising Nefopam and pharmaceutically acceptable inert excipients.

### SUMMARY OF THE INVENTION

To achieve the foregoing and other objects and needs, the present invention provides an orally disintegrating pharmaceutical composition comprising Nefopam and pharmaceutically acceptable inert excipients and a process for preparing the same.

In an embodiment, the present invention provides an orally disintegrating pharmaceutical composition comprising Nefopam and pharmaceutically acceptable inert excipients, wherein the composition comprises 5 to 10% w/w of Nefopam with respect to the total weight of the composition.

In another embodiment, the present invention provides an orally disintegrating tablet comprising:

a. Nefopam in the form of taste masked pellets or granules;

b. about 1% to about 50% w/w Diluent;

c. about 5% to about 50% w/w Disintegrant;

d. about 0.05% to about 2% w/w of Glidant; and

e. about 0.05% to about 2% w/w of Lubricant.

wherein the % are with respect to the total weight of the tablet.

In a further embodiment, the present invention provides a process for preparing an orally disintegrating pharmaceutical composition comprising Nefopam, the process comprising the steps of:

(a) Preparing pellets or granules of Nefopam;

(b) Taste masking the pellets or granules;

(c) Preparing a blend comprising various pharmaceutically acceptable inert excipients;

(d) Mixing the blend with Nefopam pellets or granules and lubricating it;

(e) Compressing the lubricated blend to form a tablet.

### DETAILED DESCRIPTION OF THE INVENTION

The exemplary embodiments described herein detail for illustrative purposes are subject to many variations in structure and design. It should be emphasized, however, that the present invention is not limited to an orally disintegrating pharmaceutical composition comprising Nefopam and pharmaceutically acceptable inert excipients and a process or preparing the same, as shown and described. It is understood that various omissions and substitutions of equivalents are contemplated as circumstances may suggest or render expedient, but these are intended to cover the application or implementation without departing from the spirit or scope of the claims of the present invention. Also, it is to be understood that the phraseology and terminology used herein is for the purpose of description and should not be regarded as limiting.

The use of terms “including,” “comprising,” or “having” and variations thereof herein is meant to encompass the items listed thereafter and equivalents thereof as well as additional items.

Further, the terms, “a” and “an” herein do not denote a limitation of quantity, but rather denote the presence of at least one of the referenced item.

As used herein, “about” will be understood by persons of ordinary skill in the art and will vary to some extent depending upon the context in which it is used. If there are uses of the term in which are not clear to persons of ordinary skill in the art, given the context in which it is used, “about” will mean up to plus or minus 10% of the particular term.

As used herein, “treatment” or “treating” are used interchangeably. These terms refer to an approach for obtaining beneficial or desired results including but not limited to a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance.

In an embodiment, the present invention provides an orally disintegrating pharmaceutical composition comprising Nefopam and pharmaceutically acceptable inert excipients, wherein the composition comprises 5 to 10% w/w of Nefopam with respect to the total weight of the composition. Specifically, the composition comprises 5% or 5.5% or 6% or 6.5% or 7% or 7.5% or 8% or 8.1% or 8.2% or 8.3% or 8.4% or 8.5% or 8.6% or 8.7% or 8.8% or 8.9% or 9% or 9.5% or 10% w/w of Nefopam with respect to the total weight of the composition.

The term Nefopam as used herein includes all its salts, esters, derivatives, prodrugs, enantiomers, isomers, complexes, and the like. Specifically, Nefopam is in the form of Nefopam Hydrochloride salt.

Nefopam as used herein is in the form of pellets or granules. Pellets are spherical or nearly spherical, free flowing granules with a narrow size distribution. The pellets are generally produced via a pelletization process whereby a powder blend consisting of drug and excipient particles is agglomerated into spherical granules. Pellets provide with a high degree of flexibility during the design and development of oral dosage forms. They can be divided into desired dose strengths without formulation or process changes, and also be blended to deliver incompatible bioactive agents simultaneously or particles with different release profiles at the same site or at different sites within gastrointestinal tract. Pellets provide development of formulation with high degree of flexibility due to their free flowing characteristic. Various pelletization techniques include agitation, compaction, compression, extrusion-spheronization, powder or solution layering, spray drying and spray congealing and the like. The granules may be formed using the techniques like slugging or compaction or aqueous or non-aqueous wet granulation.

Specifically, the present invention uses the layering technique to prepare the Nefopam pellets or granules. It includes deposition of successive layers of Nefopam from solution, suspension or dry powder on crystals or granules of the same material or inert starter seeds. The diameter of non pareil seeds is in the range of 250-355μ. The fine particle size of the non pareil seeds doesn't cause any grittiness in tongue after the disintegration of the tablet.

Non-Pareil Seeds are spherical particles of uniform diameter that are practically inert, tasteless, and odorless. Non-Pareil seeds are primarily composed of inert substances like starch, lactose. The non-Pareil seeds work as a base upon which drugs are coated. The Non-Pareil seeds are given a protective barrier.

In an aspect of the embodiment, the pellets or granules of Nefopam are taste masked.

The taste-masking techniques are applied to mask or overcome the bitter or unpleasant taste of active pharmaceutical ingredients/drugs to achieve patient acceptability and compliance. The commonly used industrial techniques/methods of taste-masking include organoleptic methods, polymer coating, hot-melt extrusion, microencapsulation, complexation, and spray-drying.

The taste of masking of Nefopam pellets or granules is done using aqueous dispersion of poly (meth) acrylate polymer. The commercially available grades of poly (meth) acrylate polymer like Eudragit NE 30D or Eudragit E by Evonik may be used for the purpose of taste masking. These taste masking agents are insoluble in saliva and prevent Nefopam to be in contact with saliva.

By the term “pharmaceutically acceptable inert excipients”, it is meant any of the components of a pharmaceutical composition other than active ingredients and which are approved by regulatory authorities or are generally regarded as safe for human or animal use. The pharmaceutically acceptable inert excipients are used during the palletization or taste masking or coating or tableting compression.

Examples of pharmaceutically acceptable inert excipients include, but are not limited to diluents, binders, sweetening agent, flavorants, disintegrants, solvents, lubricant and glidants. A combination of excipients may also be used. The amount of excipient(s) employed will depend upon how much active agent is to be used. One excipient can perform more than one function as well. The commercially available grades of the excipients can also be used for the purpose of using in the composition according to the invention.

Diluents include, but are not limited to confectioner's sugar, compressible sugar, dextrates, dextrin, dextrose, fructose, lactitol, mannitol, sucrose, starch, lactose, xylitol, sorbitol, talc, microcrystalline cellulose, calcium carbonate, calcium phosphate dibasic or tribasic, calcium sulphate and other materials known to one ordinarily skilled in the art and mixtures thereof.

Binders include, but are not limited to, starches such as potato starch, wheat starch, corn starch (maize starch); microcrystalline celluloses; celluloses such as hydroxypropyl cellulose, hydroxyethyl cellulose, hydroxypropyl methylcellulose (HPMC), ethyl cellulose, sodium carboxymethylcellulose; natural gums like acacia, alginic acid, guar gum; liquid glucose, dextrin, povidone, syrup, polyethylene oxide, polyvinylpyrrolidone, poly-N-vinyl amide, polyethylene glycol, gelatin, poly propylene glycol, tragacanth and other materials known to one ordinarily skilled in the art and mixtures thereof.

The sweetening agents (also known as sweeteners) include, but are not limited to, aspartame, saccharin sodium, dipotassium glycyrrhizinate, Stevia, thaumatin, acesulfame K, sucralose, and other materials known to one ordinarily skilled in the art and mixtures thereof.

The flavorants include natural or synthetic flavorants. The natural flavorant may be an essential oil, oleoresin, essence, or extractive. The flavoring constituents may also be derived from a spice, fruit or fruit juice, vegetable or vegetable juice, whose significant function in the product is for flavoring purpose.

The disintegrants include, but are not limited to, starch, croscarmellose sodium, polyvinyl pyrrolidone, sodium starch glycolate, microcrystalline cellulose and other materials known to one ordinarily skilled in the art and mixtures thereof.

Solvents include, but are not limited to purified water, acetone, ethyl alcohol, isopropyl alcohol dichloromethane and other materials known to one ordinarily skilled in the art and mixtures thereof.

Lubricants may be selected from, but are not limited to, those conventionally known in the art such as Mg, Al or Ca or Zn stearate, polyethylene glycol, glyceryl behenate, mineral oil, sodium stearyl fumarate, stearic acid, hydrogenated vegetable oil, talc and other materials known to one ordinarily skilled in the art and mixtures thereof.

Glidants include, but are not limited to, silicon dioxide; magnesium trisilicate, powdered cellulose, starch, talc and tribasic calcium phosphate, calcium silicate, magnesium silicate, colloidal silicon dioxide, silicon hydrogel and other materials known to one ordinarily skilled in the art and mixtures thereof. Preferably, the glidant is colloidal silicon dioxide.

The pharmaceutical composition according to the present invention is in the form of a solid dosage form.

The present invention further relates to a present invention provides an orally disintegrating tablet comprising:

a. Nefopam in the form of taste masked pellets or granules;

b. about 1% to about 50% w/w Diluent;

c. about 5% to about 50% w/w Disintegrant;

d. about 0.05% to about 2% w/w of Glidant; and

e. about 0.05% to about 2% w/w of Lubricant

wherein the % are with respect to the total weight of the tablet.

In an aspect of the embodiment, the disintegration time of the tablet is less than 180 seconds, and preferably less than 60 seconds.

“Orally disintegrating tablet” means that the tablet disintegrates or disperses within 180 seconds as measured by the in vitro disintegration test described in US Pharmacopoeia 701, without disks. Such a disintegration test result is reasonably related to the actual disintegration time experienced by a mammal when placed in the oral cavity. The disintegration of the tablet means that the tablet shape/form is destroyed but does not necessarily mean that the entire tablet is dissolved. If coated particles of the active agent are contained within the tablet, as described hereinafter, such particles can be present on the screen and need not further disintegrate, although typically such particles are too small to be held by the screen mesh and thus are also not present as a residue on the screen. Preferably, the tablets of the present invention disintegrate in less than 60 seconds.

In a further embodiment, the present invention provides a process for preparing an orally disintegrating pharmaceutical tablet comprising Nefopam, the process comprising the steps of:

(a) Preparing pellets or granules of Nefopam;

(b) Taste masking the pellets or granules;

(c) Preparing a blend comprising various pharmaceutically acceptable inert excipients;

(d) Mixing the blend with Nefopam pellets or granules and lubricating it;

(e) Compressing the lubricated blend to form a tablet.

Mixing of the excipients can be performed in a conventional device used for mixing of powders, such as for example motionless (passive) mixers, fluidized bed, diffusion, bi-conicdiffusion, uniconic, biconic, turbular, cubic, planetary, Y-, V-shaped, and low shear or high shear mixers.

Generally the drying of the pellets or granules for example can be performed in one of the following ways: trays, fluid bed, and microwave assist/vacuum/gas stripping (one pot processing).

The description of the present invention of orally disintegrating pharmaceutical composition comprising Nefopam is further illustrated by the following non-limiting example. However, a person skilled in the art would recognize that, the specific example is intended to illustrate, not limit, the scope of the present invention.

### EXAMPLES

### Example 1: Pharmaceutical Composition According to the Present Invention

TABLE 1
Pharmaceutical composition comprising Nefopam

| Ingredients | % W/W | Mg/tablet |
|---|---|---|
| Non pareil seeds (250-355μ) | 27.71 | 97.0 |
| Nefopam hydrochloride | 8.57 | 30.0 |
| Hypromellose | 2.29 | 8.0 |
| Isopropyl Alcohol | Q.S | Q.S |
| Purified water | Q.S | Q.S |
| Eudragit NE 30D | 8.74 | 30.6 |
| Hypromellose | 2.19 | 7.65 |
| Silica, Colloidal Anhydrous | 1.93 | 6.75 |
| Purified Water* | Q.S | Q.S |
| Mannitol | 3.71 | 13.00 |
| Cellulose Microcrystalline | 30.00 | 105.00 |
| Crospovidone | 10.00 | 35.00 |
| Sucralose | 2.00 | 7.00 |
| Silica, Colloidal Anhydrous | 1.00 | 3.50 |
| Orange flavor | 0.86 | 3.00 |
| Magnesium stearate | 1.00 | 3.50 |
| Total | 100.0 | 350.00 |

The manufacturing process for the preparation of Pharmaceutical composition according to example 1 comprises various steps as per paragraph [0037].

Specifically, the manufacturing process comprises the steps of,

a) Preparation of drug pellets comprising the steps of
1. Hypromellose was added under stirring in isopropyl alcohol to form a solution;
2. Nefopam hydrochloride was added to the solution formed in step a. to obtain a clear solution.
3. Purified water was added to the clear solution to form a drug loading suspension.
4. The Non pareil seeds (250-350μ) were charged in fluidized air bed coater and the drug loading suspension was added to it. The pellets were formed in this step.
5. The drug pellets were dried and sifted and milled.

The next step in the manufacturing of composition comprises the step of, b) Taste mask coating comprising the steps of
1. Hypromellose was added to purified water under continuous stirring to form a solution. Silica, colloidal anhydrous was added to the above solution to form a suspension.
2. Eudragit NE 30D was added to the suspension under stirring.
3. The pre-sifted pellets in step a) were charged in fluidized air bed coated and masked with the suspension in step 2.
4. The taste masked pellets were dried in suitable dryer.
5. The dried pellets were sifted and milled to obtain suitable pellet size.

The next step in the manufacturing of composition comprises the step of, c) preparation of a blend comprising the steps of
1. Mannitol 200+Microcrystalline Cellulose+Sucralose+Crospovidone+Silica colloidal Anhydrous+Orange flavor were sifted, through suitable sifter to form a colour blend.
2. The pre sifted taste masked pellets were transferred to suitable blended along with the colour blend formed in step 1.
3. Magnesium stearate was sifted separately.
4. The blend formed in step b. was lubricated using sifted magnesium stearate.

The next step in the manufacturing of composition comprises the step of, d) lubrication of a blend comprising the step of
1. The blend formed in step b. was lubricated using sifted magnesium stearate.

The next step in the manufacturing of composition comprises the step of, e) tablet compression comprising the step of
1. Lubricated blend formed in step d. was compressed using suitable parameters using following parameters

| SN | Parameters | Standard | |
|---|---|---|---|
| 1 | Appearance | White to off white round flat beveled | |
| | | edge tablets with central concave | |
| | | depression on both the surfaces | |
| | | having orange odour. | |
| 2 | Weight of 20 tablets | 7.00 | g |
| 3 | Average weight | 350 | mg |
| | of tablet | | |
| 4 | Thickness | 4.0 | mm |

| | | |
|---|---|---|
| 5 | Hardness | 35N |
| 6 | Disintegration time | NMT 3 Minutes at 37 ± 2° C., without disc |
| 7 | Friability | NMT 1.0% |

Nefopam Hydrochloride orally disintegrating tablets to be packed in ALU-ALU blister pack using blister packing machine.

### Example 2: Pharmaceutical Composition According to the Present Invention

TABLE 2
Pharmaceutical composition comprising Nefopam

| Ingredients | % w/w | Mg/tablet |
|---|---|---|
| Non pareil seeds (250-355μ) | 27.71 | 97.0 |
| Nefopam hydrochloride | 8.57 | 30.0 |
| Hypromellose | 2.29 | 8.0 |
| Isopropyl Alcohol | Q.S | Q.S |
| Purified water | Q.S | Q.S |
| Eudragit E | 8.74 | 30.6 |
| Hypromellose | 2.19 | 7.65 |
| Silica, Colloidal Anhydrous | 1.93 | 6.75 |
| Purified Water* | Q.S | Q.S |
| Mannitol | 3.71 | 13.00 |
| Cellulose Microcrystalline | 30.00 | 105.00 |
| Crospovidone | 10.00 | 35.00 |
| Sucralose | 2.00 | 7.00 |
| Silica, Colloidal Anhydrous | 1.00 | 3.50 |
| Orange flavor | 0.86 | 3.00 |
| Magnesium stearate | 1.00 | 3.50 |
| Total | 100.0 | 350.00 |

The composition as described in example 2 was manufactured using the process as provided in the example 1.

We claim:

1. An orally disintegrating pharmaceutical composition comprising Nefopam and pharmaceutically acceptable inert excipients, wherein the composition comprises 5 to 10% w/w of Nefopam with respect to weight of the composition.

2. The pharmaceutical composition according to claim 1, wherein the composition is in the form of a tablet.

3. The pharmaceutical composition according to claim 1, wherein the Nefopam is in the form of pellets or granules in the composition.

4. The pharmaceutical composition according to claim 3, wherein the pellets or granules are taste masked.

5. The pharmaceutical composition according to claim 3, wherein the pellets or granules contain non pareil seeds.

6. The pharmaceutical composition according to claim 5, wherein the diameter of non pareil seeds is in the range of 250-355μ.

7. An orally disintegrating tablet comprising:
a. Nefopam in the form of taste masked pellets or granules;
b. about 1% to about 50% w/w Diluent;
c. about 5% to about 50% w/w Disintegrant;
d. about 0.05% to about 2% w/w of Glidant; and
e. about 0.05% to about 2% w/w of Lubricant
wherein the % are with respect to the total weight of the tablet.

8. The tablet according to claim 7, wherein the taste masked pellets or granules comprise aqueous dispersion of poly (meth) acrylate polymer.

9. The tablet according to claim 7, wherein the diluent is sugar, dextrates, dextrin, dextrose, fructose, lactitol, mannitol, sucrose, starch, lactose, xylitol, sorbitol, talc, microcrystalline cellulose, calcium carbonate, calcium phosphate dibasic or tribasic or calcium sulphate.

10. The tablet according to claim 7, wherein the disintegrant is starch, croscarmellose sodium, polyvinyl pyrrolidone, sodium starch glycolate or microcrystalline cellulose

11. The tablet according to claim 7, wherein the taste masking of the pellets or granules is done using aqueous dispersion of poly (meth) acrylate polymer.

12. The tablet according to claim 7, wherein the disintegration time of the tablet is less than 180 seconds, and preferably less than 60 seconds.

13. A process for preparing an orally disintegrating pharmaceutical tablet comprising Nefopam, the process comprising the steps of:
(a) Preparing pellets or granules of Nefopam;
(b) Taste masking the pellets or granules;
(c) Preparing a blend comprising various pharmaceutically acceptable inert excipients;
(d) Mixing the blend with Nefopam pellets or granules and lubricating it;
(e) Compressing the lubricated blend to form a tablet.
